(19) United States
(12) Patent Application Publication
(10) Pub. No.: US 2023/0405619 A1
(43) Pub. Date: Dec. 21, 2023

(54) DISPERSION NOZZLE FOR CHEMICAL APPLICATOR

(71) Applicant: GROUP AG, LLC, Rexburg, ID (US)

(72) Inventors: Kent WASDEN, Rexburg, ID (US); Ryan WASDEN, Rexburg, ID (US)

(21) Appl. No.: 18/333,316

(22) Filed: Jun. 12, 2023

Related U.S. Application Data
(60) Provisional application No. 63/366,305, filed on Jun. 13, 2022.

(51) Int. Cl. B05B 7/16; A01G 13/06

(52) U.S. Cl. CPC B05B 7/1626; A01G 13/06

(57) ABSTRACT

A thermal fogger includes an air-supply system and a chemical injector. The air-supply system includes an air chamber, a pre-heater configured to heat air in the air chamber, and a blower in fluid communication with the air chamber and configured to blow a flow of heated air through an outlet of the air chamber. The chemical injector is coupled to the outlet of the air chamber and is configured to inject a liquid chemical into the flow of heated air to produce an air-chemical mixture.

### CROSS-REFERENCE TO RELATED APPLICATIONS

This application claims the benefit of U.S. Provisional Application No. 63/366,305, filed Jun. 13, 2022; the entire contents of this application is hereby incorporated by reference herein.

### BACKGROUND

Aerosolized chemicals may be used for agricultural purposes, such as treating crops pre-harvest or post-harvest. For example, potatoes, onions, other root vegetables may be treated with aerosolized chemicals, i.e. sprout inhibitors, while in storage to increase the freshness.

Thermal foggers may be used to aerosolize the chemical before treating the crops. Some thermal foggers use the heat from the hot gas or air to vaporize the chemical and/or drip a liquid chemical onto a hot plate so that the hot air passes over and mixes with the created vapor. These methods, however, require large amounts of energy to be at a high enough temperature to vaporize the chemical.

### SUMMARY

A thermal fogger may include an air-supply system, a chemical injector, and a heated aerosolization nozzle. The air-supply system may include an air chamber, a pre-heater configured to heat air in the air chamber, and a blower in fluid communication with the air chamber. The blower may be configured to blow a flow of heated air through an outlet of the air chamber. The chemical injector may be coupled to the outlet of the air chamber. The chemical injector may be configured to inject a chemical into the flow of heated air to produce an air-chemical mixture. The heated aerosolization nozzle may be coupled to the outlet of the air chamber and may be in fluid communication with the air chamber and the chemical injector to receive the air-chemical mixture.

In some embodiments, the heated aerosolization nozzle may include a nozzle body and a heater. The nozzle body may be shaped to define an inlet end coupled to the outlet of the air chamber, an outlet end spaced apart axially from the inlet end relative to an axis of the nozzle body, and a plurality of helical aerosolization channels that extend around the axis of the nozzle body. The heater may be arranged around an annular outer wall of the nozzle body.

In some embodiments, the plurality of helical aerosolization channels may be configured to force the air-chemical mixture flowing into the inlet end of the aerosolization nozzle radially outwards into contact with an annular outer wall of the nozzle body as the air-chemical mixture flows from the inlet end to the outlet end. The heater may be configured to heat the annular outer wall of the nozzle body to heat the air-chemical mixture in contact with the annular outer wall of the nozzle body. In this way, the air-chemical mixture may be vaporized as the air-chemical mixture flows through the plurality of helical aerosolization channels from the inlet end to the outlet end of the aerosolization nozzle. The air-chemical mixture may then be dispersed as a chemical vapor at the outlet end of the aerosolization nozzle.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 is a diagrammatic view of a thermal fogger adapted for creating aerosols from a liquid chemical showing the thermal fogger comprises an air-supply system, a chemical injector coupled air-supply system to inject a liquid chemical into a flow of heated air from the air-supply system to produce an air-chemical mixture, and a heated aerosolization nozzle coupled to the outlet of the air chamber and in fluid communication with the air chamber and the chemical injector to receive the air-chemical mixture, and further showing the heated aerosolization nozzle includes a nozzle body shaped to define a plurality of helical aerosolization channels that extend around an axis of the nozzle body to force the air-chemical mixture flowing into the inlet end of the nozzle body radially outwards into contact with an annular outer wall of the nozzle body body and a heater arranged around the annular outer wall of the nozzle body to heat the air-chemical mixture in contact with the annular outer wall of the nozzle body so that the air-chemical mixture is vaporized as it flows through the plurality of helical aerosolization channels and is dispersed as a chemical vapor at an outlet end of the nozzle body;

FIG. 2 is a perspective view of the nozzle body included in the heated aerosolization nozzle of the thermal fogger of FIG. 1 showing the nozzle body includes outer and inner walls that each extend around the axis and axially between an inlet end and an outlet end of the nozzle and a plurality of helical flow dividers that each extend between and interconnect the annular outer wall and the inner wall to define the plurality of helical aerosolization channels;

FIG. 3 is a cross-section view of the nozzle body of FIG. 2 taken along line 3-3 showing the nozzle has an inlet section, a diverging section, and an outlet section, and further showing the outer wall has a constant diameter at the inlet and outlet section, while the inner wall has a varying diameter at the diverging and outlet sections moving downstream along the axis of the nozzle body from the inlet end to the outlet end of the nozzle body such that the plurality of helical aerosolization channels have a varying cross-sectional area moving downstream along the axis of the nozzle body from the inlet end to the outlet end of the nozzle body;

FIG. 3A is a detail view of FIG. 3 showing the outer wall is comprised of two layers of different metallic materials;

FIG. 4 is a cross-section view of the nozzle body of FIG. 2 taken along line 4-4 showing the plurality of helical aerosolization channels at the inlet section of the nozzle body;

FIG. 4A is a detail view of FIG. 4 showing the plurality of helical aerosolization channels have an inlet cross-sectional area at the inlet section of the nozzle body;

FIG. 5 is a cross-section view of the nozzle body of FIG. 2 taken along line 5-5 showing the plurality of helical aerosolization channels have a greater cross-sectional area at the diverging section of the nozzle than at the inlet section of the nozzle;

FIG. 6 is a cross-section view of the nozzle body of FIG. 2 taken along line 6-6 showing the cross-sectional area of the plurality of helical aerosolization channels at the diverging section of the nozzle varies moving downstream along the axis of the nozzle body from the inlet end to the outlet end of the nozzle body;

FIG. 7 is a cross-section view of the nozzle body of FIG. 2 taken along line 7-7 showing the plurality of helical aerosolization channels have an outlet cross-sectional area at the outlet section of the nozzle;

FIG. 8 is a cross-section view of the nozzle body of FIG. 2 taken along line 8-8 showing the outlet cross-sectional area has increased from the outlet cross-sectional area shown in FIG. 7;

FIG. 9 is a cross-section view of the nozzle body of FIG. 2 taken along line 9-9 showing the outlet cross-sectional area has increased from the outlet cross-sectional area shown in FIG. 8; and

FIG. 10 is a cross-section view of the nozzle body of FIG. 2 taken along line 10-10 showing the outlet cross-sectional area has increased from the outlet cross-sectional area shown in FIG. 9 such that the greatest cross-sectional area it at the outlet end of the nozzle.

### DETAILED DESCRIPTION

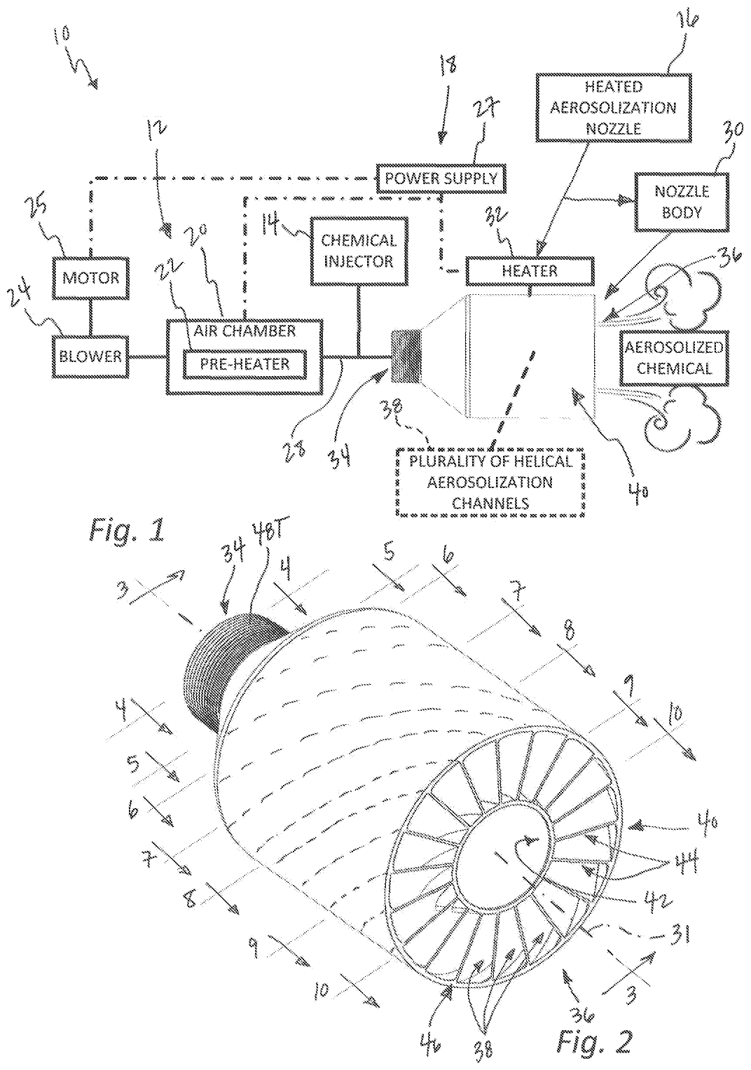

An illustrative thermal fogger 10 is shown in FIG. 1. The thermal fogger includes an air-supply system 12, a chemical injector 14, and a heated aerosolization nozzle 16 as shown in FIG. 1. The air-supply system 12 includes an air chamber 20, a pre-heater 22 configured to heat air in the air chamber 20, and a blower 24 in fluid communication with the air chamber 20 to blow a flow of heated air through an outlet 28 of the air chamber 20. The chemical injector 14 is coupled to the outlet 28 of the air chamber 20 and configured to inject a liquid chemical into the flow of heated air to produce an air-chemical mixture. The aerosolization nozzle 16 is coupled to the outlet 28 of the air chamber. The aerosolization nozzle 16 is in fluid communication with the air chamber 20 and the chemical injector 14 to receive the air-chemical mixture.

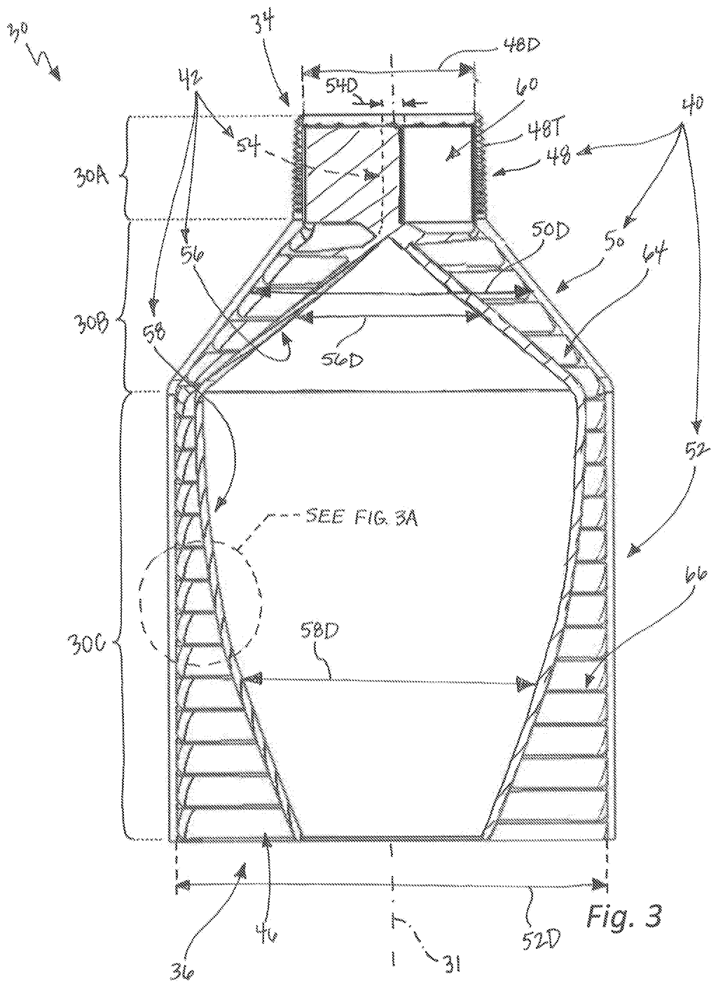

The aerosolization nozzle 16 includes a nozzle body 30 and a heater 32 as shown in FIGS. 1 and 2. The nozzle body 30 is shaped to define an inlet end 34 coupled to the outlet 28 of the air chamber 20, an outlet end spaced apart axially from the inlet end relative to an axis 31 of the nozzle body 30, and a plurality of helical aerosolization channels 38 extend around the axis 31 of the nozzle body 30. The heater 32 is arranged around an annular outer wall of the nozzle body 30.

Other thermal foggers use the heat from the heated air to vaporize the chemical and/or drip a liquid chemical onto a hot plate so that the heated air passes over and mixes with the created vapor. These methods, however, require large amounts of energy to be at a high enough temperature to vaporize the liquid chemical.

The present disclosure includes the heated aerosolization nozzle 16 to vaporize the air-chemical mixture as it flows through the nozzle body 30. The plurality of helical aerosolization channels 38 are configured to force the air-chemical mixture flowing into the inlet end 34 of the nozzle body 30 radially outwards into contact with the annular outer wall of the nozzle body 30 as the air-chemical mixture flows from the inlet end 34 to the outlet end 36. The heater 32 is configured to heat the annular outer wall 40 of the nozzle body 30 to heat the air-chemical mixture in contact with the annular outer wall 40 of the nozzle body 30. In this way, the air-chemical mixture is vaporized as it flows through the plurality of helical aerosolization channels 38 from the inlet end 34 to the outlet end 36 of the nozzle body 30 along the outer wall. The vaporized air-chemical mixture is then dispersed as a chemical vapor at the outlet end 36 of the nozzle body 30.

By heating the outer wall 40 of the nozzle body 30, the thermal fogger 10 operates at a lower energy consumption than other thermal foggers. Less energy is needed/used to heat the air from the air chamber 20, and instead heat is applied through heating the nozzle body 30. The helical aerosolization channels 38 ensure that the air-chemical mixture contacts with the heated outer wall 40.

In the illustrative embodiment, the thermal fogger 10 includes a control system 18 as shown in FIG. 1. The control system 18 is configured to control the heat applied by the pre-heater 22 and the heater 32 so that the respective heater 22, 32 heat the air or the air-chemical mixture to the desired temperature. The control system 18 includes a motor 25 and a power supply 27. The motor is coupled to the blower 24 to drive the blower 24. The power supply 27 is coupled to the pre-heater 22 and the heater 32 the provide power thereto. In the illustrative embodiment, the power supply 27 is coupled to the motor 25.

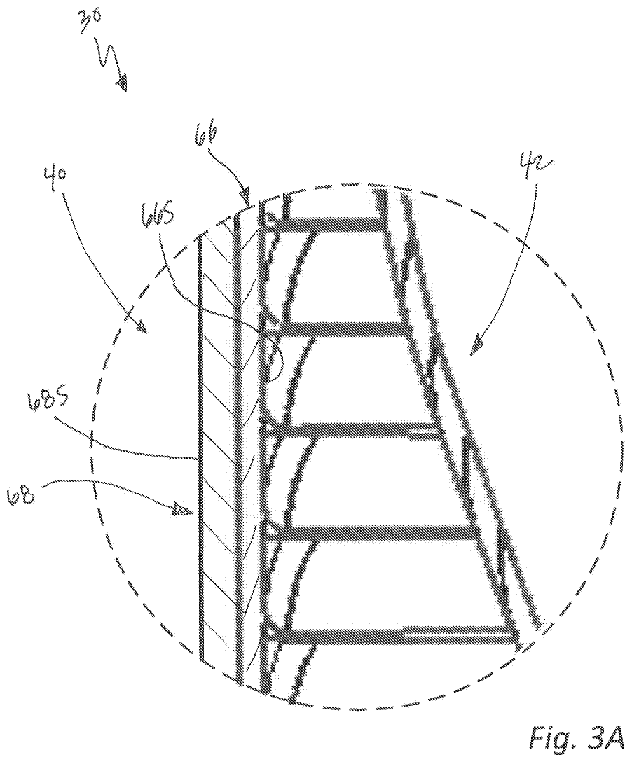

Turning again to the nozzle body 30, the nozzle body 30 has an inlet section 30A, a diverging section 30B, and an outlet section 30C as shown in FIG. 3. The inlet section 30A extends from the inlet end 34. The diverging section 30B extends axially from the inlet section 30A. The outlet section 30C extends axially from the diverging section 30B to the outlet end 36.

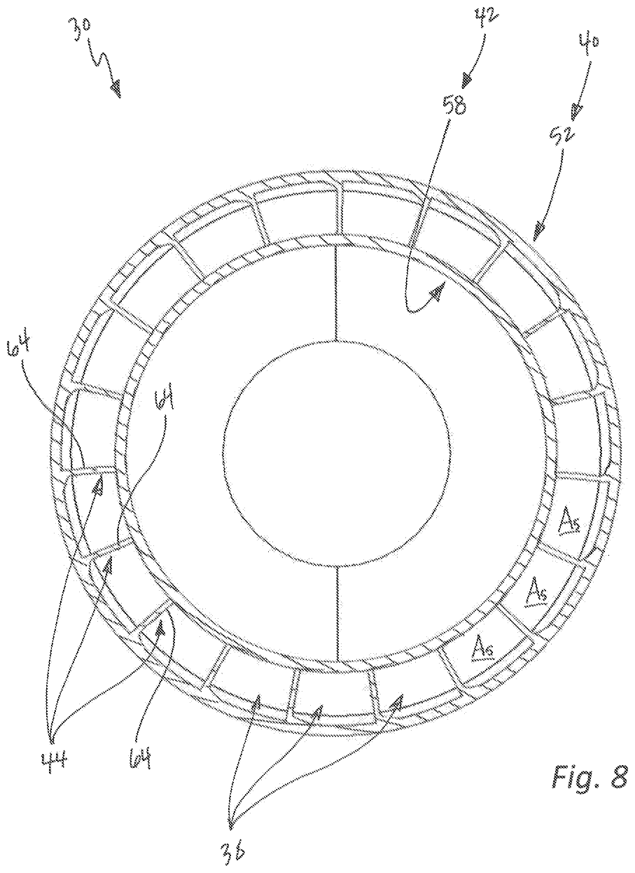

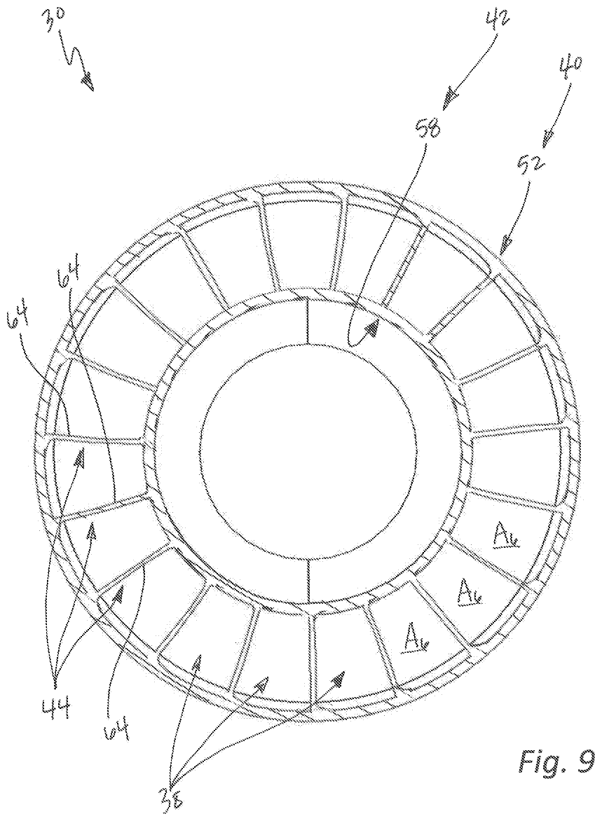

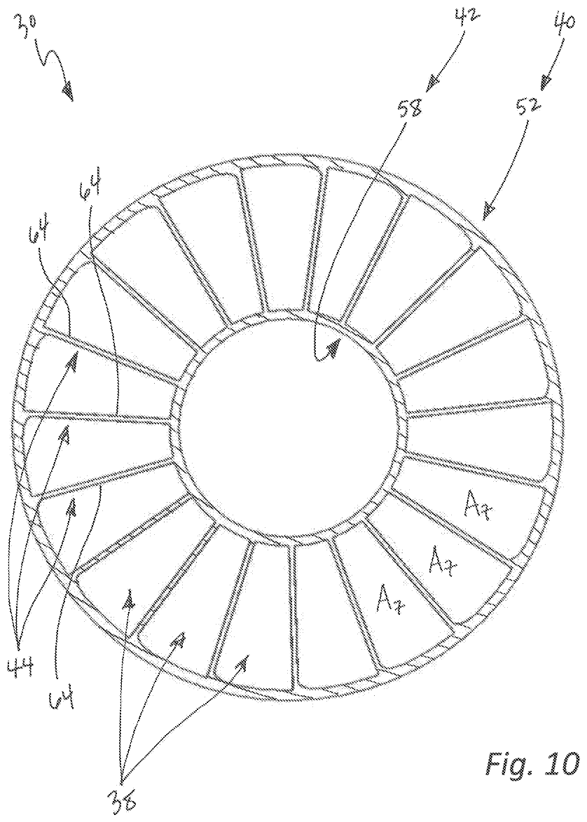

The nozzle body 30 includes the annular outer wall 40, an inner wall 42, and a plurality of helical flow dividers 44 as shown in FIGS. 2-10. The outer wall 40 and the inner wall 42 both extend around the axis 31. The inner wall 42 is located radially inward of the annular outer wall 40 to define an aerosolization chamber 46 therebetween. The plurality of helical flow dividers 44 each extend between and interconnect the annular outer wall 40 and the inner wall 42 to divide the aerosolization chamber 46 into the plurality of helical aerosolization channels 38.

In the illustrative embodiment, each flow divider 44 of the plurality of helical flow dividers 44 extends about one rotation about the axis 31. On rotation about the axis 31 is equal to 360 degrees. In other embodiments, each flow divider 44 may extend more than rotation about the axis 31.

In the illustrative embodiment, the outer wall 40 has a constant diameter 52D, while the inner wall 42 has a varying diameter 58D moving downstream along the axis 31 of the nozzle body 30 from the inlet end 34 to the outlet end 36 of the nozzle body 30. The inner wall 42 has a varying diameter 58D such that the plurality of helical aerosolization channels 38 have a varying cross-sectional area moving downstream along the axis 31.

The outer wall 40 is shaped to include an outer inlet section 48, an outer diverging section 50, and an outer outlet section 52 as shown in FIG. 3. The outer inlet section 48 corresponds to the inlet section 30A of the nozzle body 30 and extends from the inlet end 34. The outer diverging section 50 corresponds to the diverging section 30B of the nozzle body 30 and extends axially from the outer inlet section 48. The outer outlet section 52 corresponds to the outlet section 30C of the nozzle body 30 and extends axially from the outer diverging section 50 to the outlet end 36.

The outer inlet section 48 and the outer outlet section 52 have constant diameters 48D, 52D, while the outer diverging section 50 has a varying diameter 50D as shown in FIG. 3. The outer inlet section 48 has a constant diameter 48D. The outer diverging section 50 has a varying diameter 50D that increases moving axially downstream from the inlet end 34 to the outlet end 36 of the nozzle body 30. The outer outlet section 52 has a constant diameter 52D.

In the illustrative embodiment, the outer inlet section 48 is formed with threads 48T that mate with corresponding threads on the outlet 28 of the air chamber 20. The threads 48T on the outer inlet section 48 mate with corresponding threads on the outlet 28 of the air chamber 20 to couple the nozzle body 30 to the air chamber 20. In other embodiments, the outer inlet section 48 may be coupled to the outlet 28 of the air chamber 20 using a different method, such as welding.

In the illustrative embodiment, the heater 32 is arranged around the outer outlet section 52 of the annular outer wall 40. In other embodiments, the heater 32 is around the outer diverging section 50 and the outer outlet section 52 of the annular outer wall 40.

The inner wall 42 is shaped to include an inner inlet section 54, an inner diverging section 56, and an inner outlet section 58 as shown in FIG. 3. The inner inlet section 54 corresponds to the inlet section 30A of the nozzle body 30 and extends from the inlet end 34. The inner diverging section 56 corresponds to the diverging section 30B of the nozzle body 30 and extends axially from the inner inlet section 54. The inner outlet section 58 corresponds to the outlet section 30C of the nozzle body 30 and extends axially from the inner diverging section 56 to the outlet end 36.

The inner inlet section 54 has a constant diameter 54D, while the inner diverging section 56 and the inner outlet section 58 have varying diameters 56D, 58D as shown in FIG. 3. The inner inlet section 54 has a constant diameter 54D. The inner diverging section 56 has a varying diameter 56D that increases moving axially from the inlet end 34 to the outlet end 36 of the nozzle body 30. The inner outlet section 58 has a varying diameter 58D that decreases moving axially from the inlet end 34 to the outlet end 36 of the nozzle body 30.

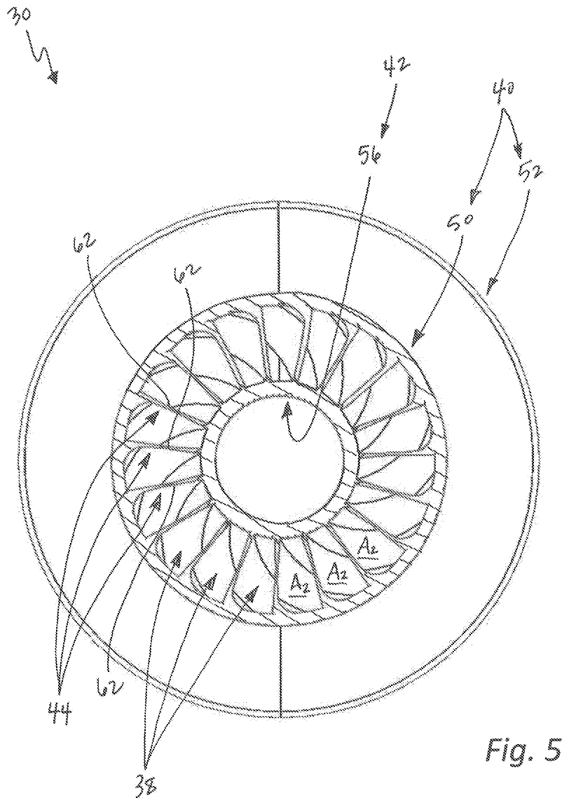

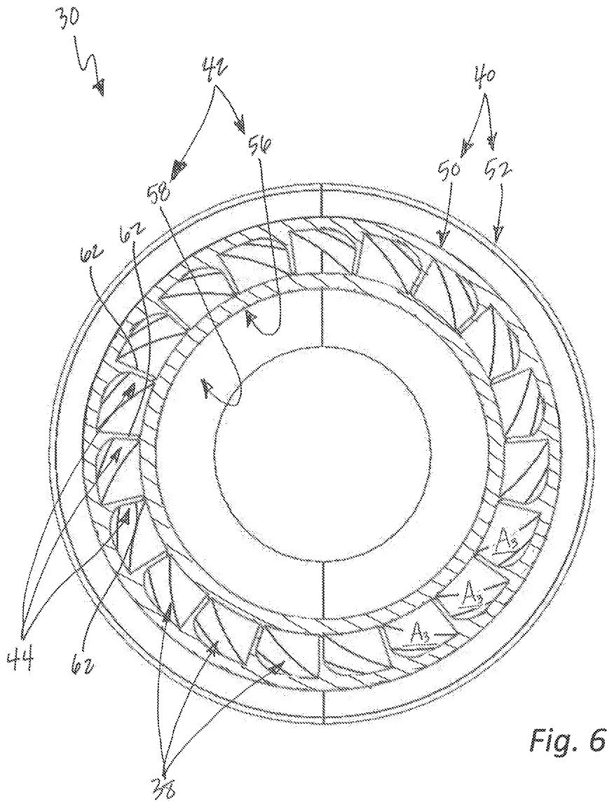

The rate at which the diameter 56D of the inner diverging section 56 varies may be different from the rate at which the diameter 50D of the outer diverging section 50 varies as shown in FIG. 3. Therefore, the cross-section shape of the helical aerosolization channels 38 in the diverging section 30B of the nozzle body 30 changes when moving along the axis 31 as shown in FIGS. 5 and 6.

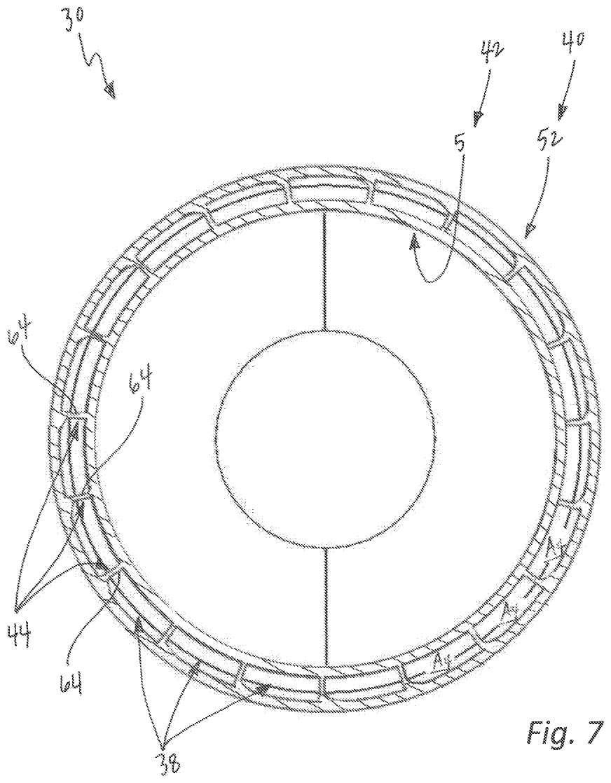

Additionally, the varying diameter 56D of the inner diverging section 56 linearly increases, while the varying diameter 58D of the inner outlet section 58 exponentially decreases as shown in FIG. 3. Therefore, the cross-section shape of the helical aerosolization channels 38 in the outlet section 30C of the nozzle body 30 changes when moving along the axis 31 as shown in FIGS. 7-10. In the illustrative embodiment, the cross-section shape of the helical aerosolization channels 38 in the outlet section 30C of the nozzle body 30 increase in size such that the cross-sectional area increases when moving downstream along the axis 31.

Each flow divider 44 has an inlet portion 60, a diverging portion 62, and an outlet portion 64 as shown in FIG. 3. The inlet portion 60 is located between the outer and inner inlet sections 48, 54 of the outer and inner walls 40, 42. The inlet portion 60 of each flow divider 44 is planar and does not have any helical element. The diverging portion 62 extends axially from the inlet portion 60 and is located between the outer and inner diverging sections 50, 56 of the outer and inner walls 40, 42. The diverging portion 62 begins the helical shape of the flow divider 44. The outlet portion 64 extends from the diverging portion 62 and is located between the outer and inner outlet sections 52, 58 of the outer and inner walls 40, 42. The outlet portion 64 continues the helical shape to the outlet end 36 of the nozzle body 30. In the illustrative embodiment, the diverging portion 62 and the outlet portion 64 form the one rotation about the axis 31.

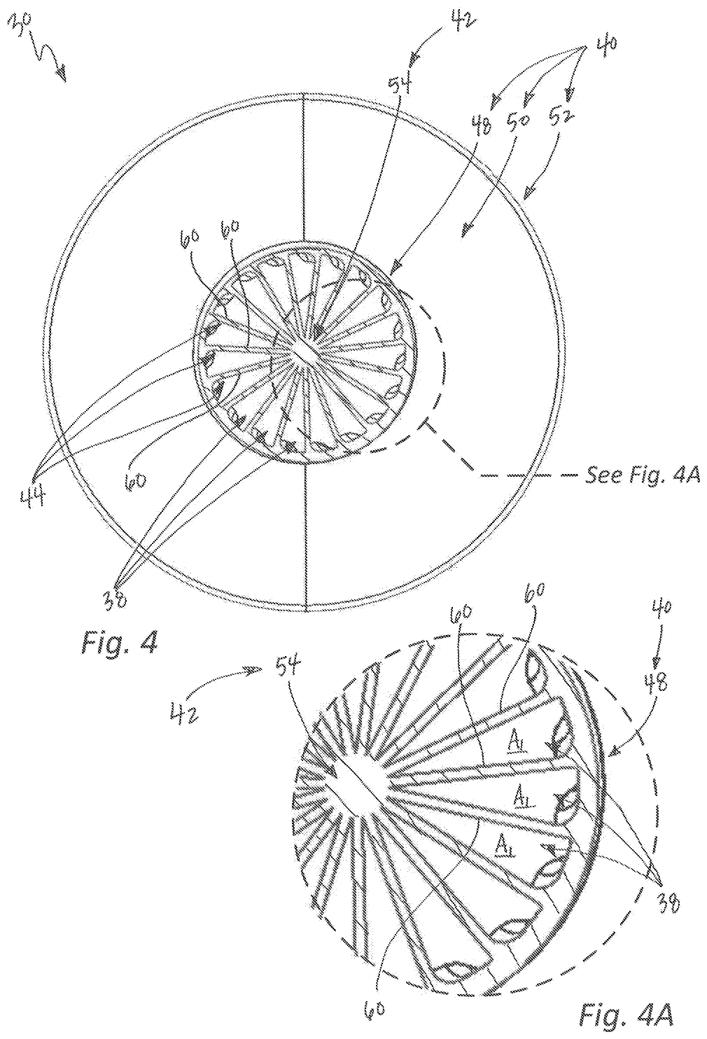

As shown in FIG. 4, each of the helical aerosolization channels 38 has an inlet cross-sectional area A₁ at a location near the inlet end 34 of the nozzle body 30. As the inlet portion 60 of each flow divider 44 is planar between the outer and inner inlet sections 48, 54 of the outer and inner walls 40, 42, the inlet cross-sectional area A₁ is constant from the inlet end 34 to the end of the outer and inner inlet sections 48, 54 of the outer and inner walls 40, 42.

As shown in FIGS. 5 and 6, each of the helical aerosolization channels 38 have a varying cross-sectional area A₂, A₃ in the diverging section 30B of the nozzle body 30. The cross-sectional area A₂, A₃ of each helical aerosolization channel 38 varies moving along the axis 31 of the nozzle body 30 from the inlet end 34 to the outlet end 36 of the nozzle body 30.

In the illustrative embodiment, the cross-sectional area A₂, A₃ of each helical aerosolization channel 38 increases moving along the axis 31. The rate at which the cross-sectional area A₂, A₃ of each helical aerosolization channel 38 increases moving along the axis 31 is linear in the illustrative embodiment.

As shown in FIGS. 7-10, each of the helical aerosolization channels 38 have a varying outlet cross-sectional area A₄, A₅, A₆, A₇ in the outlet section 30C of the nozzle body 30. The cross-sectional area A₄, A₅, A₆, A₇ of each helical aerosolization channel 38 increases moving along the axis 31 of the nozzle body 30 from the inlet end 34 to the outlet end 36 of the nozzle body 30.

In this way, the area increases as the air-chemical mixture is vaporized and expands. For example, the cross-sectional area A₄ is less than the cross-sectional area A₅, the cross-sectional area A₅ is less than the cross-sectional area A₆, and the cross-sectional area A₆ is less than the cross-sectional area A₇. The greatest cross-sectional area A₇ is at the outlet end 36 of the nozzle body 30.

In the illustrative embodiment, the nozzle body 30 is formed as a single-piece component. The nozzle body 30 may comprise metallic materials, such as stainless steel. In other embodiments, a different metallic material may be used.

In the illustrative embodiment, an additional layer of material may be added to the nozzle body 30 so that the annular outer wall 40 is comprised of two different layers 66, 68 of material as shown in FIG. 3A. The inner layer 66 may be the stainless steel material, while the outer layer 68 may be a conductive metallic material to increase the heat transfer between the heater 32 and the nozzle body 30. The inner layer 66 forms an inner surface 66S of the outer wall 40, while the outer layer 68 forms an outer surface 68S that contacts the heater 32. In this way, the stainless steel material of the inner layer 66 contacts with the air-chemical mixture, while the conductive material is in contact with the heater 32 to be heated by the heater 32.

For the purposes of the present disclosure, the modifier “about” means ±5% of a given valve. Of course, greater or lesser deviation is contemplated and may be used in processed methods within the spirit of this disclosure.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

1. A thermal fogger, comprising:
an air-supply system including an air chamber, a pre-heater configured to heat air in the air chamber, and a blower in fluid communication with the air chamber and configured to blow a flow of heated air through an outlet of the air chamber;
a chemical injector coupled to the outlet of the air chamber and configured to inject a chemical into the flow of heated air to produce an air-chemical mixture; and
a heated aerosolization nozzle coupled to the outlet of the air chamber and in fluid communication with the air chamber and the chemical injector to receive the air-chemical mixture, the heated aerosolization nozzle comprising:
a nozzle body shaped to define an inlet end coupled to the outlet of the air chamber, an outlet end spaced apart axially from the inlet end relative to an axis of the nozzle body, and a plurality of helical aerosolization channels that extend around the axis of the nozzle body, the plurality of helical aerosolization channels configured to force the air-chemical mixture flowing into the inlet end of the nozzle body radially outwards into contact with an annular outer wall of the nozzle body as the air-chemical mixture flows from the inlet end to the outlet end, and
a heater arranged around the annular outer wall of the nozzle body and configured to heat the annular outer wall of the nozzle body to heat the air-chemical mixture in contact with the annular outer wall of the nozzle body so that the air-chemical mixture is vaporized as the air-chemical mixture flows through the plurality of helical aerosolization channels from the inlet end to the outlet end of the nozzle body and dispersed as a chemical vapor at the outlet end of the nozzle body.

2. The apparatus of claim 1, wherein the plurality of helical aerosolization channels have a varying cross-sectional area moving along the axis of the nozzle body from the inlet end to the outlet end of the nozzle body.

3. The apparatus of claim 2, wherein the cross-sectional area of each channel included in the plurality of helical aerosolization channels increases moving along the axis of the nozzle body from the inlet end to the outlet end of the nozzle body.

4. The apparatus of claim 1, wherein the nozzle body includes the annular outer wall that extends around the axis, an inner wall that extends around the axis and is located radially inward of the annular outer wall to define an aerosolization chamber therebetween, and a plurality of helical flow dividers that each extend between and interconnect the annular outer wall and the inner wall to divide the aerosolization chamber into the plurality of helical aerosolization channels.

5. The apparatus of claim 4, wherein each flow divider of the plurality of helical flow dividers extends one rotation about the axis.

6. The apparatus of claim 5, wherein the outer wall has a constant diameter and the inner wall has a varying diameter moving along the axis of the nozzle body from the inlet end to the outlet end of the nozzle body.

7. The apparatus of claim 1, wherein the nozzle body has an inlet section that extends from the inlet end, a diverging section that extends axially from the inlet section, and an outlet section that extends axially from the diverging section to the outlet end, and wherein the outer wall has a constant diameter at the inlet section, a varying diameter at the diverging section, and a constant diameter at the outlet section that is greater than the constant diameter at the inlet section.

8. The apparatus of claim 1, wherein the heater is an induction heater.

9. The apparatus of claim 1, wherein the chemical injected by the chemical injector is a liquid chemical.

10. A heated aerosolization nozzle adapted to aerosolize an air-chemical mixture, the heated aerosolization nozzle comprising
a nozzle body shaped to define an inlet end, an outlet end spaced apart axially from the inlet end relative to an axis of the nozzle body, and a plurality of helical aerosolization channels that extend around the axis of the nozzle body, the plurality of helical aerosolization channels configured to force the air-chemical mixture flowing into the inlet end of the nozzle body radially outwards into contact with an annular outer wall of the nozzle body as the air-chemical mixture flows from the inlet end to the outlet end, and
a heater arranged around the annular outer wall of the nozzle body and configured to heat the annular outer wall of the nozzle body to heat the air-chemical mixture in contact with the annular outer wall of the nozzle body so that the air-chemical mixture is vaporized as it flows through the plurality of helical aerosolization channels from the inlet end to the outlet end of the nozzle body and dispersed as a chemical vapor at the outlet end of the nozzle body.

11. The apparatus of claim 10, wherein the plurality of helical aerosolization channels have a varying cross-sectional area moving along the axis of the nozzle body from the inlet end to the outlet end of the nozzle body.

12. The apparatus of claim 11, wherein the cross-sectional area of each channel included in the plurality of helical aerosolization channels increases moving along the axis of the nozzle body from the inlet end to the outlet end of the nozzle body.

13. The apparatus of claim 10, wherein the nozzle body includes the annular outer wall that extends around the axis, an inner wall that extends around the axis and is located radially inward of the annular outer wall to define an aerosolization chamber therebetween, and a plurality of helical flow dividers that each extend between and interconnect the annular outer wall and the inner wall to divide the aerosolization chamber into the plurality of helical aerosolization channels.

14. The apparatus of claim 13, wherein each flow divider of the plurality of helical flow dividers extends one rotation about the axis.

15. The apparatus of claim 13, wherein the outer wall has a constant diameter and the inner wall has a varying diameter moving along the axis of the nozzle body from the inlet end to the outlet end of the nozzle body.

16. The apparatus of claim 10, wherein the heater is an induction heater.

17. The apparatus of claim 10, wherein the chemical injected by the chemical injector is a liquid chemical.

18. A method of aerosolizing a chemical
providing an air-supply system including an air chamber, a chemical injector coupled to an outlet of the air chamber and configured to inject a liquid chemical, and a heated aerosolization nozzle coupled to the outlet of the air chamber and in fluid communication with the air chamber and the chemical injector, the heated aerosolization nozzle comprising a nozzle body shaped to define a plurality of helical aerosolization channels that extend around an axis of the nozzle body,
heating air within the air chamber,
directing a flow of heated air within the air chamber through the outlet of the air chamber into an inlet end of the nozzle body,
injecting the liquid chemical into the flow of heated air as the flow of heated air flow toward the inlet end of the nozzle body to produce an air-chemical mixture,
applying heat to an outer wall of the nozzle body, and
directing the air-chemical mixture flowing into the inlet end of the nozzle body into the plurality of helical aerosolization channels to force the air-chemical mixture radially outwards into contact with the heated outer wall of the nozzle body as the air-chemical mixture flows from the inlet end to the outlet end so that the air-chemical mixture is vaporized as the air-chemical mixture flows through the plurality of helical aerosolization channels from the inlet end to the outlet end of the nozzle body and dispersed as a chemical vapor at the outlet end of the nozzle body.

19. The method of claim 18, wherein the chemical injected by the chemical injector is a liquid chemical.
